(19) United States
(12) Patent Application Publication
(10) Pub. No.: US 2007/0113844 A1
(43) Pub. Date: May 24, 2007

(54) Endoscopic bite block

(76) Inventors: Mary L. Garren, Carmel, CA (US); Lloyd R. Garren, Carmel, CA (US); Silas Lum, Monterey, CA (US)

Correspondence Address: CONNOLLY BOVE LODGE & HUTZ, LLP, P O BOX 2207, WILMINGTON, DE, 19899, US

(21) Appl. No.: 11/285,050

(22) Filed: Nov. 22, 2005

(51) Int. Cl. A61M 16/00

(52) U.S. Cl. 128/200.26

(57) ABSTRACT

A bite block intended primarily for use with upper gastrointestinal endoscopy. The bite block comprises a unitary fabrication that fits into the mouth between the teeth or dental ridges and has a central passageway large enough to accommodate a gastroscope. The bite block also has a surface which lies exterior to the oral cavity and extends around the outer surface of the lips. The bite block also has a suction device which can be attached to a standard suction machine via flexible tubing or with an intermediary hollow wand. The suction device extends through the bite block into the intra-oral portion thereof where it forms an angle and allows suction drainage of pooled oral fluids from the cheek cavity. The interior tip of the suction device has multiple slit like openings circumferentially arranged to allow suction drainage of oral fluids without becoming occluded by the oral tissues.

### BACKGROUND OF THE INVENTION

The present invention relates to an endoscopic bit block, and more particularly to a bite block that comprises a unitary body which allows for easy passage of an endoscope and which includes an integrated mouth suction angled in a manner that catches and removes pooled oral fluids that accumulate during endoscopic procedures.

Instruments, such as endoscopes, are inserted through a person's mouth into the human body, in medical procedures. To prevent the patient from biting into the instrument, a bite block is used which is generally a plastic tube positioned in the patient's open mouth. The instrument is then inserted through the bite block opening into the stomach or other areas.

Presently, endoscopy is usually performed with the patient in the left lateral position using a bite block which maintains the teeth apart to protect the endoscope from damage by the teeth. This method makes it difficult to suction secretions and increases the risk of aspiration and potential procedure related complications. The currently available bite blocks require a second person and apparatus to accomplish oral suction. In addition, access for oral suction is difficult to obtain with currently available bite blocks. Bite blocks of one type or another are shown in U.S. Des. 283,158; U.S. Des 329,908; U.S. Des. 348,932; U.S. Pat. No. 4,270,529; U.S. Pat. No. 4,351,331; U.S. Pat. No. 4,425,911; U.S. Pat. No. 4,495,945; U.S. Pat. No. 4,646,273; U.S. Pat. No. 5,009,227; U.S. Pat. No. 5,174,284; U.S. Pat. No. 5,305,742; U.S. Pat. No. 5,413,095 and U.S. Pat. No. 6,257,238.

### SUMMARY OF THE INVENTION

One of the objects of the present invention is to provide a bite block for endoscopy with a self-contained suction ability in a unitary device.

Another object of the present invention is an endoscopic bite block that enables mouth suction without obstruction by oral tissues.

Still another object of the invention is an endoscopic bite block which is simple in construction, but highly effective and efficient in gastrointestinal endoscopy procedures.

Basically, the bite block of the present invention is extremely useful for performing endoscopy. The block provides protection for the endoscope while simultaneously allowing the evacuation of pooled intra-oral secretions without the labor of an additional person.

Fundamentally, the bite block of the present invention comprises a unitary body fabricated from plastic that is rigid enough to withstand pressure from apposition of the mandible to maxilla and having an exterior peri-labial component which is flexible enough to slightly mold to the exterior of the lips. The labial component has strap retainers at the sides thereof to allow attachment of an elastic strap for securing for the bite block (via an elastic strap around the head connecting one side to the other). In addition, the bite block has a suction device extending from the exterior labial surface to the intra-oral surface. The suction device at the labial surface has the ability to be connected to a commercial suction source by flexible tubing or with an intermediary hollow suction wand. The terminal intra-oral portion of the suction device has an angle which allows suctioning where secretions pool during endoscopy which is usually conducted with the patient in the left lateral position. In addition, the terminal portion of the intra-oral suction device has slits circumferentially located and surrounding a circular terminal opening. This design prevents complete occlusion of the suction device by oral tissues.

In accordance with the present invention, an endoscopic bite block for a person undergoing upper gastrointestinal endoscopy comprises a unitary body having an intra-oral portion and an exterior portion. A central passageway extends through the unitary body, and the passageway is constructed and arranged to receive an endoscope. A suction device extends through the unitary body from the exterior to the intra-oral portions thereof, and the suction device includes an angled intra-oral end generally pointing to the check cavity of the person undergoing gastrointestinal endoscopy for suction removal of pooled oral fluids.

Preferably, the angled intra-oral end of the suction device includes multiple circumferential slit like openings that allow suction drainage of pooled oral fluids without occlusion by the oral tissues of the person undergoing gastrointestinal endoscopy. Moreover, the central passageway and the suction device are oriented generally parallel to one another, and the angled intra-oral end of the suction device is generally transverse to such orientation.

The exterior portion of the unitary body is peri-labial and slightly flexible so as to mold to the exterior of the lips of the person undergoing gastrointestinal endoscopy. Moreover, the bite block may include an elastic strap, the ends of which are connected to the exterior portion of the unitary body for securement around the head of the person undergoing gastrointestinal endoscopy.

In accordance with the present invention, the unitary body of the endoscopic bite block includes a suction passageway extending therethrough from the exterior to the intra-oral portions thereof, and the suction device extends through the suction passageway. A releasable snap fit connection may be provided between the suction device and the unitary body.

### BRIEF DESCRIPTION OF THE DRAWING

Novel features and advantages of the present invention in addition to those mentioned above will become apparent to persons of ordinary skill in the art from a reading of the following detailed description in conjunction with the accompanying drawings wherein similar reference characters refer to similar parts and in which:

FIG. 1 is a perspective view of an endoscopic bite block, according to the present invention;

FIG. 2 is a front elevational view of the endoscopic bite block shown in FIG. 1;

FIG. 3 is a left side elevational view of the endoscopic bite block shown in FIG. 1;

FIG. 4 is a right side elevational view of the endoscopic bite block shown in FIG. 1;

FIG. 5 is a rear elevational review of the endoscopic bite block shown in FIG. 1;

FIG. 6 is a top plan view of the endoscopic bite block shown in FIG. 1;

FIG. 7 is a top plan view similar to FIG. 6 showing an elastic strap for holding the block in the mouth of the user and also showing details of the suction device of the block; and

FIG. 8 is a side elevational view of an alternate suction wand for use with the bite block.

### DETAILED DESCRIPTION OF THE INVENTION

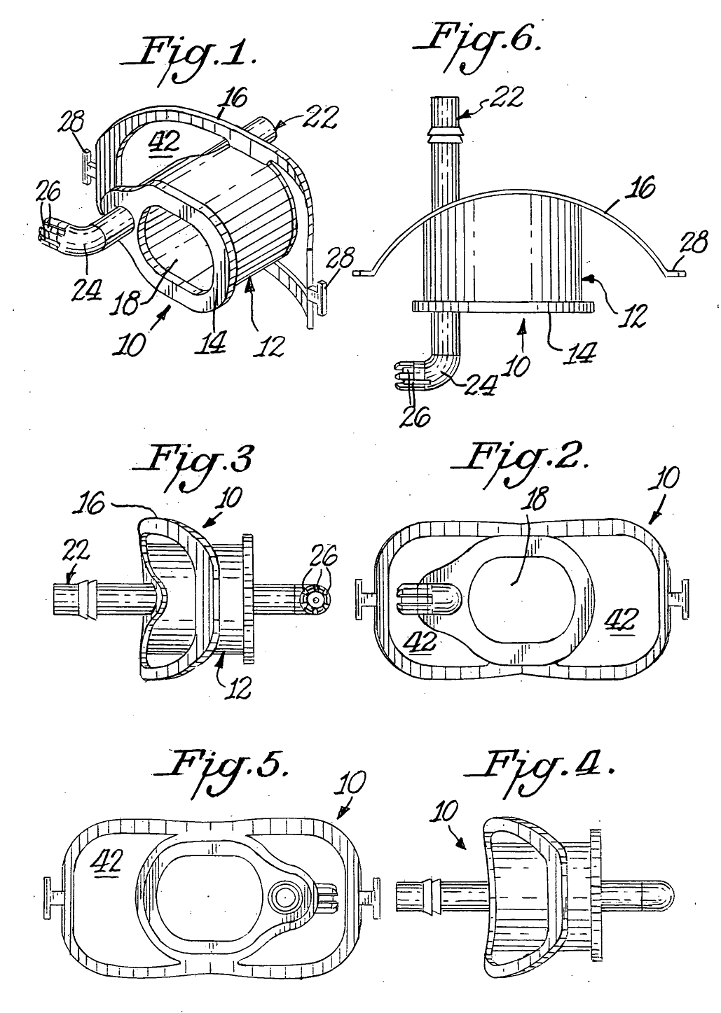

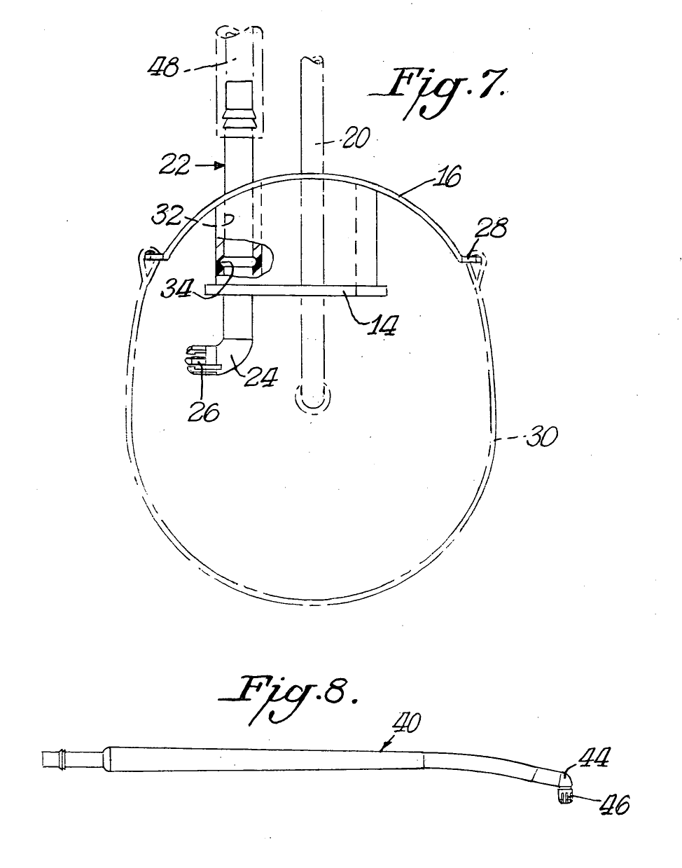

Referring in more particularity to the drawings, FIG. 1-6 illustrate an endoscopic bite block 10 for a person undergoing upper gastrointestinal endoscopy. The bite block comprises a unitary body 12 having an intra-oral portion 14 and an exterior portion 16. The unitary body may be formed from thermoplastic material by molding techniques known in the art. A central passageway 18 extends through the unitary body 12, and the passageway is arranged to receive an endoscope 20, such as shown in FIG. 7.

A suction device 22 extends through the unitary body 12 from the exterior portion 16 to the intra-oral portion 14 of the unitary body 12. The suction device 22 includes an angled intra-oral end 24 generally pointing to the left check cavity of a person undergoing gastrointestinal endoscopy for the suction removal of pooled oral fluids.

The angled intra-oral end 24 of the suction device 22 includes multiple circumferential slit like openings 26 that allow suction drainage of pooled oral fluids without occlusion thereof by the oral tissues of the person undergoing gastrointestinal endoscopy.

The central passageway 18 through the unitary body 12 of the bite block 10 and the suction device 22 are oriented generally parallel to one another, and the angled intra-oral end 24 of the suction device is generally transverse to such orientation. Fundamentally, the angled intra-oral end 24 of the suction device is formed at a right angle to the balance of the device, and as such the angled end 24 points to the left check cavity of the person undergoing gastrointestinal endoscopy. Normally the person lies on the left side during these procedures whereby the suction device is properly positioned for suction drainage of pooled oral fluids in the left cheek cavity. However, should the person lie on the right side the block is easily inverted which then points the intra-oral end 24 of the suction device 22 to the right.

The exterior portion 16 of the unitary body 12 is peri-labial and slightly flexible so as to mold to the exterior of the lips of the person undergoing gastrointestinal endoscopy. Strap retainers 24 are positioned on the right and left sides of the exterior portion 16 of the unitary body 12, and an elastic strap 30 is connected to the retainers 28 for securement around the head of the person undergoing gastrointestinal endoscopy. This particular feature of the invention is specifically shown in FIG. 7.

Also, as best shown in FIG. 7, the unitary body 12 includes a suction passageway extending therethrough from the exterior portion 16 to the intra-oral portion 14. The suction device 22 extends through the suction passageway 32. A releasable snap fit connection 34 may be provided between the suction device and the unitary body. Removal of the suction device from the unitary body may be accomplished by simply withdrawing the suction device from the passageway 32, if desired. As an alternative to the snap fit connection 34, a friction fit may be utilized where the suction device 22 simply frictionally engages the walls of passageway 32.

FIG. 8 illustrates an aspirating wand 40 which may be used in place of the suction device 22 by simply extending the wand through the suction passageway 32 of the unitary body 12 of the bite block 10. Also, the wand may be used in combination with the suction device 22 by passing the wand through the open spaces 42 in the exterior portion 16 of the unitary body 12. The wand may be manipulated to remove pooled oral fluids. The wand preferably includes an angled end 44 with multiple circumferential slit like openings 46 therein.

Both the suction device 22 and the wand 40 are connected by tubing 48 is a suction source (not shown).

What is claimed is:

1. An endoscopic bite block for a person undergoing upper gastrointestinal endoscopy, the bite block comprising a unitary body having an intra-oral portion and an exterior portion, a central passageway extending through the unitary body constructed and arranged to receive an endoscope, a suction device extending through the unitary body from the exterior to the intra-oral portions thereof, and the suction device including an angled intra-oral end generally pointing to the check cavity of a person undergoing gastrointestinal endoscopy for the suction removal of pooled oral fluids.

2. An endoscopic bite bock as in claim 1 wherein the angled intra-oral end of the suction device includes multiple circumferential slit like openings that allow suction drainage of pooled oral fluids without occlusion by oral tissues of a person undergoing gastrointestinal endoscopy.

3. An endoscopic bite block as in claim 1 wherein the central passageway and the suction device are oriented generally parallel to one another, and the angled intra-oral end of suction device is generally transverse to such orientation.

4. An endoscopic bite block as in claim 1 wherein the exterior portion of the unitary body is peri-labial and slightly flexible so as to mold to the exterior of the lips of a person undergoing gastrointestinal endoscopy.

5. An endoscopic bite bock as in claim 1 including with elastic strap the ends of which are connected to the exterior portion of the unitary body for securement around the head of a person undergoing gastrointestinal endoscopy.

6. An endoscopic bite block as in claim 1 wherein the unitary body includes a suction passageway extending therethrough from the exterior to the intra-oral portions thereof, and wherein the suction device extends through the suction passageway.

7. An endoscopic bite block as in claim 6 including a releasable snap fit connection between the suction device and the unitary body.
